(19) United States
(12) Patent Application Publication
(10) Pub. No.: US 2026/0217634 A1
(43) Pub. Date: Jul. 30, 2026

(54) REDUCING GAS FLARING THROUGH CONVERTING FLOWBACK FLUIDS INTO E-FUELS AND CHEMICALS

(71) Applicant: SAUDI ARABIAN OIL COMPANY, Dhahran (SA)

(72) Inventors: Ghassan S. ALSHEHRY, Dhahran (SA); Hassan Sakar ALQAHTANI, Dhahran (SA); Khalid Mohammed ALRUWAILI, Dhahran (SA)

(73) Assignee: SAUDI ARABIAN OIL COMPANY, Dhahran (SA)

(21) Appl. No.: 19/040,543

(22) Filed: Jan. 29, 2025

(51) Int. Cl. C07C 29/151; C01B 3/06; C01B 3/32; C07C 51/00; E21B 43/34

(52) U.S. Cl. CPC C07C 29/1518; C01B 3/06; C01B 3/32; C07C 51/00; E21B 43/34; C01B 2203/06; C01B 2203/1241; C01B 2203/141

(57) ABSTRACT

A method for processing flowback may comprise: providing flowback fluid comprising produced water, methane, hydrogen sulfide, and carbon dioxide; separating the flowback fluid into produced water, methane, hydrogen sulfide, carbon dioxide streams; producing a hydrogen stream and a carbon dioxide stream from the methane stream; producing a hydrogen stream from the hydrogen sulfide stream; and producing a hydrogen stream from the produced water stream. A flowback fluid processing system may comprise: a flowback fluid separator; a water splitting unit; a methane conversion unit; a hydrogen sulfide converter; a carbon dioxide reducing unit; and a synthesis unit.

### FIELD

The present disclosure generally relates to methods and systems for processing flowback fluid and converting it into valuable chemicals.

### BACKGROUND

As the world aims to reduce greenhouse gas (GHG) emissions to create a sustainable world, scientists are trying to tackle high emitting sources, one of which involves upstream operations. In a 2022 report by the International Energy Agency (IEA), upstream operations (production and transportation) contributed to 15% of total GHG emissions. In the early stage of production, the well goes through a flowback stage, an essential stage for clean-up, removal of fracking material, and optimizing well production. This stage could last anywhere between one to three months. During the flowback stage, flowback fluid flows back through the wellhead. Flowback fluid usually includes produced water, fracking materials, sand, methane, hydrogen sulfide, and carbon dioxide. The flowback fluid flowing back through the wellhead initially has a large proportion of fracking materials. As time goes on, the flowback fluid's proportion of materials transitions to less fracking material and more oil/gas. Eventually, the flowback stage ends and the well is put into regular production.

During the flowback stage, methane can be flared for clean-up and the early stage of the well's production. This flaring contributes to considerable amounts of carbon dioxide accumulating in the atmosphere, which is an environmental concern. The target for IEA's net zero emissions (NZE) is to eliminate all non-emergency flaring. Consequently, there is a desire to capture and utilize flowback, especially for unconventional wells, such as those located in Jafurah Basin, which is a large unconventional natural gas field.

### SUMMARY

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

In one or more aspects, the present disclosure provides a method for processing flowback comprising: providing flowback fluid comprising produced water, methane, hydrogen sulfide, and carbon dioxide; separating the flowback fluid into produced water, methane, hydrogen sulfide, carbon dioxide streams; producing a hydrogen stream and a carbon dioxide stream from the methane stream; producing a hydrogen stream from the hydrogen sulfide stream; and producing a hydrogen stream from the produced water stream.

In another aspect, the present disclosure provides A flowback fluid processing system may comprise: a flowback fluid separator; a water splitting unit; a methane conversion unit; a hydrogen sulfide converter; a carbon dioxide reducing unit; and a synthesis unit.

Any combinations of the various embodiments, aspects, and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

The FIGURE shows a schematic flow diagram of an embodiment of a system and method of the present disclosure.

### DETAILED DESCRIPTION

The present disclosure generally relates to methods and systems for processing flowback fluid and converting it into valuable chemicals.

The present disclosure provides methods related to using well bore flowback fluids to produce synthetic fuels and chemicals. The methods disclosed herein may separate flowback fluids into produced water, methane, hydrogen sulfide, and carbon dioxide, convert them into hydrogen and carbon monoxide, and utilize the hydrogen and carbon monoxide to produce valuable chemicals through Fisher-Tropsch synthesis and other processes. Among the advantages that these methods offer includes producing valuable chemicals and synthetic fuels from flowback fluid, thus contributing towards a net zero emissions future and eliminating or reducing gas emissions. The methods specifically can prevent non-emergency flaring and instead utilize natural gas rather than flaring it. A further advantage is the separation each component from the flowback fluids, which components are difficult to separate by known methods.

In one embodiment, the methods of this disclosure for processing flowback fluid comprise: providing a flowback fluid the comprises produced water, methane, hydrogen sulfide, carbon dioxide; separating the flowback fluid into produced water, methane, hydrogen sulfide, carbon dioxide streams; producing a hydrogen stream and a carbon dioxide stream from the methane stream; producing a hydrogen stream from the hydrogen sulfide stream; and producing a hydrogen stream from the produced water stream. The methods may include separating fracking materials out of the flowback fluid before separating the flowback fluid into produced water, methane, hydrogen sulfide, and carbon dioxide streams. The carbon dioxide stream may be converted to carbon monoxide and combined with the hydrogen streams to form syngas. This syngas may then be converted to various synthesis products such as light hydrocarbons or heavier hydrocarbons including naptha, gasoline, and diesel.

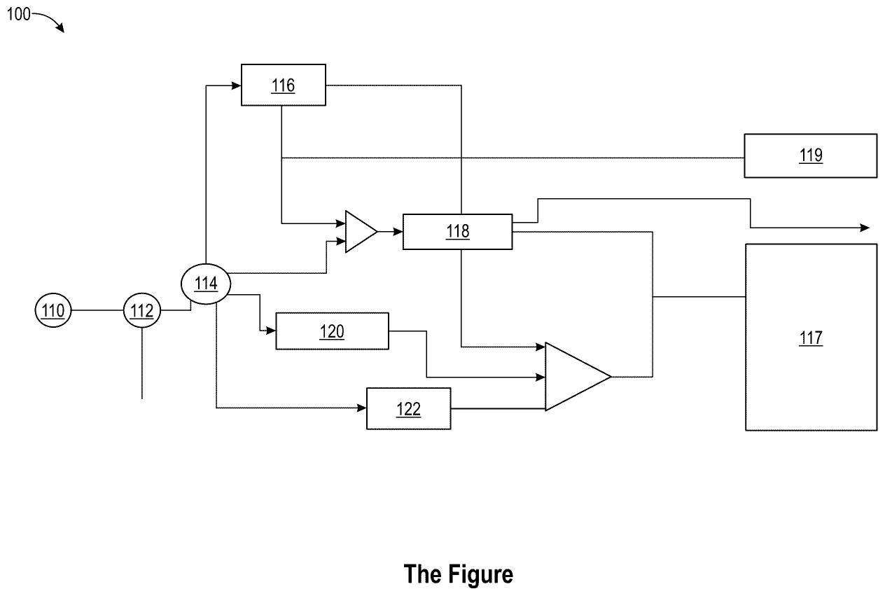

The methods of this disclosure will now be discussed with reference to FIG. 1. Shown in FIG. 1 is a schematic of a process 100 according to the present disclosure. The process begins at wellhead 110. At wellhead 110, a flowback fluid is recovered. This flowback fluid may include produced water, hydrocarbons, hydrogen sulfide, carbon dioxide, fracking materials, nitrogen, helium, other gases, the like, and combinations thereof. Fracking materials may include solids, proppant, fracking chemicals, or the like. Hydrocarbons may include methane and medium to long hydrocarbons. The content of the flowback fluid may depend on the location of the well. Optionally, the flowback fluid may recovered in a tank or other suitable container for storage until processing. Flowback fluid may flow from wellhead 110 to fracking materials separator 112. Fracking materials separator 112 may separate fracking materials from the flowback fluid. Fracking materials separator 112 may include filters and/or centrifuges to separate out fracking materials from the flowback fluid.

The flowback fluid may flow from fracking materials separator 112 to flowback fluid separator 114. Flowback fluid separator 114 may separate flowback fluid into a methane stream, a carbon dioxide stream, a hydrogen sulfide stream, and a produced water stream. These streams may include small amounts of other compounds. Flowback fluid separator 114 may separate methane from medium to long hydrocarbons if short and medium to long hydrocarbons are present in the flowback fluid. The methane stream may include from about 70 mol % methane to about 90 mol % methane, based on the mol % of the methane stream.

The methane stream may flow to methane conversion unit 116. Methane conversion unit 116 may convert the methane stream into a hydrogen gas stream and carbon dioxide stream. Methane conversion unit 116 may perform this conversion by steam methane reformation, dry methane reformation, plasma reformation, partial oxidation of methane, autothermal reformation, catalytic methane decomposition, the like, or combinations thereof. The hydrogen gas stream from the methane conversion unit 116 may be combined with the carbon monoxide stream to produce syngas and flowed to synthesis unit 117.

The carbon dioxide stream from the methane conversion unit 116 may be flowed to carbon dioxide reducing unit 118 for reduction or flowed to mineralization unit 119 for mineralization to carbon minerals such as calcium minerals, calcite, magnesium minerals, magnesite, and the like. Mineralization unit 119 may produce carbon minerals by mineralization by using at least some of the carbon dioxide stream from methane conversion unit 116. Mineralization may include in-situ or ex-situ mineralization. In-situ mineralization may include injecting the carbon dioxide stream into subterranean formations for storage and/or for reaction with reactive rocks (e.g., basalt). Ex-situ mineralization may include carbonation of minerals including magnesium silicates, calcium silicates, carbonation of industrial waste including fly ash, cement kiln dust, steel slag, aqueous carbonation including dissolving carbon dioxide in water and reacting with metal ions, the like, or combinations thereof.

The carbon dioxide stream from flowback fluid separator 114 may be fed to carbon dioxide reducing unit 118 for reduction or to mineralization unit 119. Carbon dioxide reducing unit 118 may reduce carbon dioxide to carbon monoxide or formic acid. Carbon dioxide reducing unit 118 may reduce carbon dioxide to formic acid or carbon monoxide by reverse water-gas shift reactions, chemical reduction with a catalyst and hydrogen gas, electrochemical reduction, catalysis techniques, or the like. The hydrogen gas used by carbon dioxide reducing unit 118 may be sourced from the hydrogen gas produced by process 100 as opposed to externally provided hydrogen gas. The formic acid produced carbon dioxide reducing unit 118 may be flowed to storage or used in another chemical method. The carbon monoxide stream from carbon dioxide reducing unit 118 may be combined with the hydrogen streams of this disclosure to make syngas and flowed to synthesis unit 117.

The hydrogen sulfide stream from flowback fluid 114 may be converted to a hydrogen stream and sulfur by hydrogen sulfide converter 120. Hydrogen sulfide converter 120 may perform this conversion via decomposition processes, a Claus process (conversion to sulfur and sulfuric acid and then further processed to hydrogen and sulfur), catalytic decomposition, electrocatalysis, photocatalysis, thermal cracking, the like, or combinations thereof. The hydrogen from hydrogen sulfide converter 120 may be combined with the carbon monoxide stream of this disclosure and flowed to synthesis unit 117. The sulfur from hydrogen sulfide converter 120 may be collected.

The produced water stream from flowback fluid separator 114 may be flowed to water splitting unit 122. Water splitting unit 122 may convert produced water to a hydrogen stream and oxygen via electrolysis, photocatalysis, the like, or combinations thereof. The oxygen may be stored or used in another chemical method. The hydrogen stream may be combined with the carbon monoxide stream to form syngas and flowed to synthesis unit 117. Each hydrogen stream of this disclosure may be combined with the carbon monoxide stream to form syngas and flowed to synthesis unit 117. The ratio of carbon monoxide to hydrogen may be adjusted before flowing to synthesis unit 117.

Synthesis unit 117 may include reactors and catalyst beds for converting syngas into synthesis products. Synthesis products include methanol, ethanol, mixed alcohols, ammonia, dimethyl ether, Fischer-Tropsch liquids, and other fuels or chemicals. Process 100 may not include venting or flaring methane or hydrogen sulfide. Flaring, and grammatical equivalents thereof used herein, is a step in a process where the burning of gases including natural gas, hydrogen sulfide, or other gases occurs so as to prevent the gas from escaping into the atmosphere. The synthesis unit may source the hydrogen and carbon monoxide it uses solely from process 100.

### Non-Limiting Clauses

The present disclosure is further directed to the following non-limiting embodiments:
Clause 1: A method for processing flowback comprising: providing flowback fluid comprising produced water, methane, hydrogen sulfide, and carbon dioxide; separating the flowback fluid into produced water, methane, hydrogen sulfide, carbon dioxide streams; producing a hydrogen stream and a carbon dioxide stream from the methane stream; producing a hydrogen stream from the hydrogen sulfide stream; and producing a hydrogen stream from the produced water stream.
Clause 2: The method of clause 1, further comprising reducing the carbon dioxide stream from the step for separating the flowback fluid and the carbon dioxide stream from the step for producing a carbon dioxide stream from the methane stream, to produce a carbon monoxide stream.
Clause 3: The method of clauses 1 or 2, further comprising combining the hydrogen streams with the carbon monoxide stream to form syngas.
Clause 4: The method of any of clauses 1-3, further comprising converting the syngas to synthesis products.
Clause 5: The method of any of clauses 1-4, wherein the method does not comprise flaring the methane stream.
Clause 6: The method of any of clauses 1-5, further comprising mineralizing at least some of the carbon dioxide stream produced from the methane stream to carbon minerals.
Clause 7: The method of any of clauses 1-6, wherein mineralizing comprises ex-situ mineralization.
Clause 8: The method of any of clauses 1-7, wherein mineralizing comprises in-situ mineralization.
Clause 9: The method of any of clauses 1-8, wherein the methane stream comprises from about 70 mol % methane to about 90 mol % methane, based on the mol % of the methane stream.
Clause 10: The method of any of clauses 1-9, further comprising reducing the carbon dioxide stream from the step for separating the flowback fluid and the carbon dioxide stream from the step for producing a carbon dioxide stream from the methane stream to produce a formic acid.
Clause 11: The method of any of clauses 1-10, wherein reducing the carbon dioxide streams comprises at least one of a reverse water-gas shift reaction and electrochemical reduction.
Clause 12: The method of any of clauses 1-11, wherein the syngas used to produce synthesis products is solely derived from the flowback fluid.
Clause 13: The method of any of clauses 1-12, wherein producing a hydrogen stream and a carbon dioxide stream from the methane stream is performed by at least one of steam methane reformation, dry methane reformation, partial oxidation, and catalytic methane decomposition.
Clause 14: The method of any of clauses 1-14, further comprising separating fracking materials out of the flowback fluid before separating the flowback fluid into produced water, methane, hydrogen sulfide, and carbon dioxide streams.
Clause 15: A flowback fluid processing system comprising: a flowback fluid separator; a water splitting unit; a methane conversion unit; a hydrogen sulfide converter; a carbon dioxide reducing unit; and a synthesis unit.
Clause 16: The system of clause 15, further comprising a mineralization unit.
Clause 17: The system of clause 15 or 16, wherein the flowback fluid separator separates carbon dioxide from the flowback fluid.
Clause 18: The system of any of clauses 15-17, further comprising producing carbon monoxide via the carbon dioxide reducing unit.
Clause 19: The system of any of clauses 15-18, further comprising producing formic acid via the carbon dioxide reducing unit.
Clause 20: The system of any of clauses 15-19, wherein a methane stream is fed to the methane conversion unit and the methane stream comprises from about 70 mol % methane to about 90 mol % methane, based on the mol % of the methane stream.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element, or group of elements is preceded with the transitional phrase “comprising,” it is understood that we also contemplate the same composition or group of elements with transitional phrases “consisting essentially of,” “consisting of,” “selected from the group consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related, and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

What is claimed is the following:

1. A method for processing flowback comprising:
providing flowback fluid comprising
produced water,
methane,
hydrogen sulfide, and
carbon dioxide;
separating the flowback fluid into produced water, methane, hydrogen sulfide, carbon dioxide streams;
producing a hydrogen stream and a carbon dioxide stream from the methane stream;
producing a hydrogen stream from the hydrogen sulfide stream; and
producing a hydrogen stream from the produced water stream.

2. The method of claim 1, further comprising reducing the carbon dioxide stream from the step for separating the flowback fluid and the carbon dioxide stream from the step for producing a carbon dioxide stream from the methane stream, to produce a carbon monoxide stream.

3. The method of claim 2, further comprising combining the hydrogen streams with the carbon monoxide stream to form syngas.

4. The method of claim 3, further comprising converting the syngas to synthesis products.

5. The method of claim 1, wherein the method does not comprise flaring the methane stream.

6. The method of claim 2, further comprising mineralizing at least some of the carbon dioxide stream produced from the methane stream to carbon minerals.

7. The method of claim 6, wherein mineralizing comprises ex-situ mineralization.

8. The method of claim 6, wherein mineralizing comprises in-situ mineralization.

9. The method of claim 1, wherein the methane stream comprises from about 70 mol % methane to about 90 mol % methane, based on the mol % of the methane stream.

10. The method of claim 1, further comprising reducing the carbon dioxide stream from the step for separating the flowback fluid and the carbon dioxide stream from the step for producing a carbon dioxide stream from the methane stream to produce a formic acid.

11. The method of claim 2, wherein reducing the carbon dioxide streams comprises at least one of a reverse water-gas shift reaction and electrochemical reduction.

12. The method of claim 4, wherein the syngas used to produce synthesis products is solely derived from the flowback fluid.

13. The method of claim 1, wherein producing a hydrogen stream and a carbon dioxide stream from the methane stream is performed by at least one of steam methane reformation, dry methane reformation, partial oxidation, and catalytic methane decomposition.

14. The method of claim 1, further comprising separating fracking materials out of the flowback fluid before separating the flowback fluid into produced water, methane, hydrogen sulfide, and carbon dioxide streams.

15. A flowback fluid processing system comprising:
a flowback fluid separator;
a water splitting unit;
a methane conversion unit;
a hydrogen sulfide converter;
a carbon dioxide reducing unit; and
a synthesis unit.

16. The system of claim 15, further comprising a mineralization unit.

17. The system of claim 15, wherein the flowback fluid separator separates carbon dioxide from the flowback fluid.

18. The system of claim 15, further comprising producing carbon monoxide via the carbon dioxide reducing unit.

19. The system of claim 15, further comprising producing formic acid via the carbon dioxide reducing unit.

20. The system of claim 15, wherein a methane stream is fed to the methane conversion unit and the methane stream comprises from about 70 mol % methane to about 90 mol % methane, based on the mol % of the methane stream.
